(19) United States
(12) Patent Application Publication
(10) Pub. No.: US 2009/0258084 A1
(43) Pub. Date: Oct. 15, 2009

(54) Mono and Combination Therapy with a M1/M4 Muscarinic Agonist (Sabcomeline) for Treatment of Cognitive Disorders in Schizophrenia

(76) Inventors: Paul Christopher Sharpe, Essex (GB); Peter Robin Blower, Essex (GB); Jill Galloway Chisnall Rasmussen, Essex (GB)

Correspondence Address: NIXON & VANDERHYE, PC, 901 NORTH GLEBE ROAD, 11TH FLOOR, ARLINGTON, VA, 22203, US

(21) Appl. No.: 12/226,431

(22) PCT Filed: Apr. 23, 2007
(86) PCT No.: PCT/GB2007/001474

§ 371 Date: Jan. 15, 2009

(30) Foreign Application Priority Data
Apr. 21, 2006 (GB) 0607946.1

(51) Int. Cl. A61K 33/00; A61K 31/202; A61P 25/18

(52) U.S. Cl. 424/600; 514/560

(57) ABSTRACT

The invention relates to the use of a functional muscarinic M1/M4 receptor agonist such as the functional muscarinic M1/M4 receptor agonist or a pharmaceutically acceptable salt thereof, in particular sabcomeline or a pharmaceutically acceptable salt thereof in for the treatment of cognitive impairment in schizophrenia and to combination therapies for the treatment of cognitive impairment in schizophrenia which the functional muscarinic M1/M4 receptor agonist or a pharmaceutically acceptable salt thereof, in particular sabcomeline or a pharmaceutically acceptable salt thereof and at least one other neuroprotective agent and/or neuroleptic and/or atypical antipsyhcotic agent are administered adjunctively or simultaneously. The invention provides methods of treatment of cognitive impairment in schizophrenia utilising such therapies and such adjunctive or simultaneous therapeutic combination therapies, therapeutic combinations for use therein and pharmaceutical compositions comprising them.

The invention relates to the treatment of cognitive impairment in schizophrenia, to therapeutic combinations and combinations comprising them for use in the treatment of cognitive impairment in schizophrenia, and to methods of treatment of cognitive impairment in schizophrenia.

U.S. Pat. No. 5,278,170 describes a class of compounds which enhance cholinergic neuronal activity via functional action at muscarinic M1/M4 receptors within the central nervous system. A particularly preferred compound from within the scope of this disclosure has been given the common name sabcomeline. The chemical name for sabcomeline is R-(Z)-α-(methoxyimino)-α-(1-azabicyclo[2.2.2]oct-3-yl)acetonitrile. For therapeutic administration, it is preferably used in the form of a pharmaceutically acceptable salt, typically the hydrochloride salt, but alternative salts of sabcomeline with pharmaceutically acceptable acids may also be utilised in therapeutic administration, for example salts derived from sabcomeline free base and acids including, but not limited to, hydrobromic acid, phosphoric acid, acetic acid, fumaric acid, maleic acid, salicylic acid, citric acid, lactic acid, oxalic acid and p-toluene sulphonic acid.

WO 02/03684 discloses the treatment of psychotic disorders including bipolar disorders and mania by administration of a muscarinic receptor agonist in combination with a typical or an atypical antipsychotic. As referred to below, the cognitive impairment that characterises schizophrenia is the main determinant of the patient's inability to function in society, and there remains a need to identify treatments of cognitive impairment in schizophrenia, and in particular compositions and methods of treatment which improve on the efficacy of existing therapies. There is no disclosure in WO 02/03684 of any activity of any muscarinic receptor agonist combinations against cognitive impairment in schizophrenia, nor is there any enabling disclosure of how to demonstrate or optimise this activity.

Cognitive function refers to the general ability to organise, process, and recall information. Cognitive tasks may be subdivided into a large number of more specific functions, depending on the nature of the information remembered and the circumstances of its recall. In addition, there are many functions commonly associated with cognition such as the ability to execute complex sequences of tasks. Disturbances of cognitive function may occur in a variety of disorders and the precise characteristics of the cognitive impairment vary according to the disorder.

For instance in dementia the major problem is in the early stages of the disease is progressive memory loss. The most common cause of dementia is Alzheimer's Disease. Impairment of cognitive function may also occur in psychiatric disorders such as the psychoses and depression.

In the last decade it has been realised that the cognitive impairment that characterises schizophrenia is the main determinant of these patients inability to function in society. The cognitive impairment in schizophrenia is primarily associated with problems with executive function. This has now become an important and major new topic of research.

The manifestations of cognitive impairment in schizophrenia are characterised by deficits in working memory, attention/vigilance and executive function (verbal learning and memory, visual learning and memory, speed of processing, reasoning and problem solving) and social cognition. These are present from the outset of the disease and worsen progressively during the course of illness.

There remains a need to identify treatments of cognitive impairment in schizophrenia, and in particular compositions and methods of treatment which improve on the efficacy of existing therapies.

It has now been found that functional muscarinic M1/M4 receptor agonists such as sabcomeline or a pharmaceutically acceptable salt thereof may advantageously be administered as monotherapy to treat cognitive impairment in schizophrenia.

For the avoidance of doubt, it is intended that the term schizophrenia covers the full spectrum of schizophrenic disorders known to the skilled person. These include, but are not limited to, the following: catatonic, disorganised, paranoid, residual and undifferentiated schizophrenia; schizophreniform disorder and schizoaffective disorder.

In a first aspect therefore, the invention provides a method of treatment of cognitive impairment in schizophrenia by administration of a functional muscarinic M1/M4 receptor agonist. In a further aspect, the invention provides the use of a functional muscarinic M1/M4 receptor agonist in the manufacture of a medicament for the treatment of cognitive impairment in schizophrenia.

The invention also provides the use of a functional muscarinic M1/M4 receptor agonist for the treatment of cognitive impairment in schizophrenia.

The invention further provides a functional muscarinic M1/M4 receptor agonist for use in the treatment of cognitive impairment in schizophrenia.

Functional muscarinic M1/M4 receptor agonists are compounds which enhance cholinergic neuronal activity at the muscarinic M1/M4 receptors predominantly. This functional selectivity results in a level of safety and tolerability advantageous for use in the treatment of cognitive impairment in schizophrenia. Sabcomeline is one such functional muscarinic M1/M4 receptor agonist. Other suitable functional M1/M4 receptor agonists or combinations thereof may also be used.

For therapeutic administration according to the present invention, the functional muscarinic M1/M4 receptor agonist, in particular sabcomeline may be employed in the form of its free base, but is preferably used in the form of a pharmaceutically acceptable salt, typically the hydrochloride salt.

Alternative salts of the functional muscarinic M1/M4 receptor agonist, in particular sabcomeline with pharmaceutically acceptable acids may also be utilised in therapeutic administration, for example salts derived from the functional muscarinic M1/M4 receptor agonist, in particular sabcomeline free base and acids including, but not limited to, hydrobromic acid, phosphoric acid, acetic acid, fumaric acid, maleic acid, salicylic acid, citric acid, oxalic acid, lactic acid, malic acid, methanesulphonic acid and p-toluene sulphonic acid.

All solvates and all alternative physical forms of the functional muscarinic M1/M4 receptor agonist, in particular sabcomeline or its pharmaceutically acceptable derivatives as described herein, including but not limited to alternative crystalline forms, amorphous forms and polymorphs are also within the scope of this invention, and all references to the functional muscarinic M1/M4 receptor agonist, in particular sabcomeline herein include all pharmaceutically acceptable salts, and all solvates and alternative physical forms thereof.

For therapeutic administration according to the invention, the functional muscarinic M1/M4 receptor agonist or a pharmaceutically acceptable salt thereof, in particular sabcomeline or its pharmaceutically acceptable salts or solvates may be administered in pure form, but will preferably be formulated into any suitable pharmaceutically acceptable and effective composition which provides effective levels of the active ingredient in the body.

The treatment of cognitive impairment may include administering the functional muscarinic M1/M4 receptor agonist or a pharmaceutically acceptable salt thereof, in particular sabcomeline or a pharmaceutically acceptable salt thereof at a dose of between 10 μg-200 μg. Preferably, the dose is between 20 μg-100 μg. More preferably, the dose is between 25 μg-50 μg. The dose may be administered as a single dose or twice daily. Ideally, the functional muscarinic M1/M4 receptor agonist or a pharmaceutically acceptable salt thereof, in particular sabcomeline or a pharmaceutically acceptable salt thereof is administered at a dose of 25 μg twice daily.

Typically, the functional muscarinic M1/M4 receptor agonist or a pharmaceutically acceptable salt thereof, in particular sabcomeline or a pharmaceutically acceptable salt thereof is administered to the patient at dose ranges of 20 to 50 μg total daily dose with titration to optimal dose in the range 10 to 200 μg total daily dose.

Less preferably, the functional muscarinic M1/M4 receptor agonist or a pharmaceutically acceptable salt thereof, in particular sabcomeline or a pharmaceutically acceptable salt thereof or a pharmaceutically acceptable salt thereof is administered independently of any other medication.

It has also been found that sabcomeline or a pharmaceutically acceptable salt thereof may advantageously be administered in combination with at least one neuroprotective agent and/or neuroleptic agent (which may be a typical or atypical antipsychotic agent) to provide improved treatment of cognitive impairment in schizophrenia. The combinations, uses and methods of treatment of the invention may also provide advantages in treatment of patients who fail to respond adequately or who are resistant to other known treatments.

In a further aspect, the invention provides a method of treatment of cognitive impairment in schizophrenia by adjunctive therapeutic administration of the functional muscarinic M1/M4 receptor agonist or a pharmaceutically acceptable salt thereof, in particular sabcomeline or a pharmaceutically acceptable salt thereof to a patient receiving therapeutic administration of at least one neuroprotective agent and/or neuroleptic agent (which may be a typical or atypical antipsychotic agent).

In a further aspect, the invention provides a method of treatment of cognitive impairment in schizophrenia by adjunctive therapeutic administration of a functional muscarinic M1/M4 receptor agonist or a pharmaceutically acceptable salt thereof, in particular sabcomeline or a pharmaceutically acceptable salt thereof to a patient receiving therapeutic administration of at least one neuroprotective agent.

In a further aspect, the invention provides a method of treatment of cognitive impairment in schizophrenia by adjunctive therapeutic administration of a functional muscarinic M1/M4 receptor agonist or a pharmaceutically acceptable salt thereof, in particular sabcomeline or a pharmaceutically acceptable salt thereof to a patient receiving therapeutic administration of at least one neuroleptic agent.

In a further aspect, the invention provides a method of treatment of cognitive impairment in schizophrenia by adjunctive therapeutic administration of a functional muscarinic M1/M4 receptor agonist or a pharmaceutically acceptable salt thereof, in particular sabcomeline or a pharmaceutically acceptable salt thereof to a patient receiving therapeutic administration of at least one typical antipsychotic agent.

In a further aspect, the invention provides a method of treatment of cognitive impairment in schizophrenia by adjunctive therapeutic administration of a functional muscarinic M1/M4 receptor agonist or a pharmaceutically acceptable salt thereof, in particular sabcomeline or a pharmaceutically acceptable salt thereof to a patient receiving therapeutic administration of at least one atypical antipsychotic agent.

In a further aspect, the invention provides the use of a functional muscarinic M1/M4 receptor agonist or a pharmaceutically acceptable salt thereof, in particular sabcomeline or a pharmaceutically acceptable salt thereof in the manufacture of a medicament for adjunctive therapeutic administration for the treatment of cognitive impairment in schizophrenia in a patient receiving therapeutic administration of at least one neuroprotective and neuroleptic agent (which may be a typical or atypical antipsychotic agent).

In a further aspect, the invention provides the use of a functional muscarinic M1/M4 receptor agonist or a pharmaceutically acceptable salt thereof, in particular sabcomeline or a pharmaceutically acceptable salt thereof in the manufacture of a medicament for adjunctive therapeutic administration for the treatment of cognitive impairment in schizophrenia in a patient receiving therapeutic administration of at least one neuroprotective agent.

In a further aspect, the invention provides the use of a functional muscarinic M1/M4 receptor agonist or a pharmaceutically acceptable salt thereof, in particular sabcomeline or a pharmaceutically acceptable salt thereof in the manufacture of a medicament for adjunctive therapeutic administration for the treatment of cognitive impairment in schizophrenia in a patient receiving therapeutic administration of at least one neuroleptic agent.

In a further aspect, the invention provides the use of a functional muscarinic M1/M4 receptor agonist or a pharmaceutically acceptable salt thereof, in particular sabcomeline or a pharmaceutically acceptable salt thereof in the manufacture of a medicament for adjunctive therapeutic administration for the treatment of cognitive impairment in schizophrenia in a patient receiving therapeutic administration of at least one typical antipsychotic agent.

In a further aspect, the invention provides the use of a functional muscarinic M1/M4 receptor agonist or a pharmaceutically acceptable salt thereof, in particular sabcomeline or a pharmaceutically acceptable salt thereof in the manufacture of a medicament for adjunctive therapeutic administration for the treatment of cognitive impairment in schizophrenia in a patient receiving therapeutic administration of at least one atypical antipsychotic agent.

The invention also provides the use of a functional muscarinic M1/M4 receptor agonist or a pharmaceutically acceptable salt thereof, in particular sabcomeline or a pharmaceutically acceptable salt thereof in adjunctive therapeutic administration for the treatment of cognitive impairment in schizophrenia in a patient receiving therapeutic administration of at least one neuroprotective agent and/or neuroleptic agent (which may be a typical or atypical antipsychotic agent).

The invention also provides the use of a functional muscarinic M1/M4 receptor agonist or a pharmaceutically acceptable salt thereof, in particular sabcomeline or a pharmaceutically acceptable salt thereof in adjunctive therapeutic administration for the treatment of cognitive impairment in schizophrenia in a patient receiving therapeutic administration of at least one neuroprotective agent.

The invention also provides the use of a functional muscarinic M1/M4 receptor agonist or a pharmaceutically acceptable salt thereof, in particular sabcomeline or a pharmaceutically acceptable salt thereof in adjunctive therapeutic administration for the treatment of cognitive impairment in schizophrenia in a patient receiving therapeutic administration of at least one neuroleptic agent.

The invention also provides the use of a functional muscarinic M1/M4 receptor agonist or a pharmaceutically acceptable salt thereof, in particular sabcomeline or a pharmaceutically acceptable salt thereof in adjunctive therapeutic administration for the treatment of cognitive impairment in schizophrenia in a patient receiving therapeutic administration of at least one typical antipsychotic agent.

The invention also provides the use of a functional muscarinic M1/M4 receptor agonist or a pharmaceutically acceptable salt thereof, in particular sabcomeline or a pharmaceutically acceptable salt thereof in adjunctive therapeutic administration for the treatment of cognitive impairment in schizophrenia in a patient receiving therapeutic administration of at least one atypical antipsychotic agent.

The invention also provides a functional muscarinic M1/M4 receptor agonist or a pharmaceutically acceptable salt thereof, in particular sabcomeline or a pharmaceutically acceptable salt thereof for use for adjunctive therapeutic administration for the treatment of cognitive impairment in schizophrenia in a patient receiving therapeutic administration of at least one neuroprotective agent and/or neuroleptic agent (which may be a typical or atypical antipsychotic agent).

The invention also provides a functional muscarinic M1/M4 receptor agonist or a pharmaceutically acceptable salt thereof, in particular sabcomeline or a pharmaceutically acceptable salt thereof for use for adjunctive therapeutic administration for the treatment of cognitive impairment in schizophrenia in a patient receiving therapeutic administration of at least one neuroprotective agent.

The invention also provides a functional muscarinic M1/M4 receptor agonist or a pharmaceutically acceptable salt thereof, in particular sabcomeline or a pharmaceutically acceptable salt thereof for use for adjunctive therapeutic administration for the treatment of cognitive impairment in schizophrenia in a patient receiving therapeutic administration of at least one neuroleptic agent.

The invention also provides a functional muscarinic M1/M4 receptor agonist or a pharmaceutically acceptable salt thereof, in particular sabcomeline or a pharmaceutically acceptable salt thereof for use for adjunctive therapeutic administration for the treatment of cognitive impairment in schizophrenia in a patient receiving therapeutic administration of at least one typical antipsychotic agent.

The invention also provides a functional muscarinic M1/M4 receptor agonist or a pharmaceutically acceptable salt thereof, in particular sabcomeline or a pharmaceutically acceptable salt thereof for use for adjunctive therapeutic administration for the treatment of cognitive impairment in schizophrenia in a patient receiving therapeutic administration of at least one atypical antipsychotic agent. In a further aspect, the invention provides a method of treatment of cognitive impairment in schizophrenia by adjunctive therapeutic administration of at least one neuroprotective and/or neuroleptic agent (which may be a typical or atypical antipsychotic agent) to a patient receiving therapeutic administration of the functional muscarinic M1/M4 receptor agonist or a pharmaceutically acceptable salt thereof, in particular sabcomeline or a pharmaceutically acceptable salt thereof.

In a further aspect, the invention provides a method of treatment of cognitive impairment in schizophrenia by adjunctive therapeutic administration of at least one neuroprotective agent to a patient receiving therapeutic administration of a functional muscarinic M1/M4 receptor agonist or a pharmaceutically acceptable salt thereof, in particular sabcomeline or a pharmaceutically acceptable salt thereof.

In a further aspect, the invention provides a method of treatment of cognitive impairment in schizophrenia by adjunctive therapeutic administration of at least one neuroleptic agent to a patient receiving therapeutic administration of a functional muscarinic M1/M4 receptor agonist or a pharmaceutically acceptable salt thereof, in particular sabcomeline or a pharmaceutically acceptable salt thereof.

In a further aspect, the invention provides a method of treatment of cognitive impairment in schizophrenia by adjunctive therapeutic administration of at least one typical antipsychotic agent to a patient receiving therapeutic administration of a functional muscarinic M1/M4 receptor agonist or a pharmaceutically acceptable salt thereof, in particular sabcomeline or a pharmaceutically acceptable salt thereof.

In a further aspect, the invention provides a method of treatment of cognitive impairment in schizophrenia by adjunctive therapeutic administration of at least one atypical antipsychotic agent to a patient receiving therapeutic administration of a functional muscarinic M1/M4 receptor agonist or a pharmaceutically acceptable salt thereof, in particular sabcomeline or a pharmaceutically acceptable salt thereof.

In a further aspect, the invention provides the use of at least one neuroprotective and/or neuroleptic agent (which may be a typical or atypical antipsychotic agent) in the manufacture of a medicament for adjunctive therapeutic administration for the treatment of cognitive impairment in schizophrenia in a patient receiving therapeutic administration of a functional muscarinic M1/M4 receptor agonist or a pharmaceutically acceptable salt thereof, in particular sabcomeline or a pharmaceutically acceptable salt thereof.

In a further aspect, the invention provides the use of at least one neuroprotective agent in the manufacture of a medicament for adjunctive therapeutic administration for the treatment of cognitive impairment in schizophrenia in a patient receiving therapeutic administration of a functional muscarinic M1/M4 receptor agonist or a pharmaceutically acceptable salt thereof, in particular sabcomeline or a pharmaceutically acceptable salt thereof.

In a further aspect, the invention provides the use of at least one neuroleptic agent in the manufacture of a medicament for adjunctive therapeutic administration for the treatment of cognitive impairment in schizophrenia in a patient receiving therapeutic administration of a functional muscarinic M1/M4 receptor agonist or a pharmaceutically acceptable salt thereof, in particular sabcomeline or a pharmaceutically acceptable salt thereof.

In a further aspect, the invention provides the use of at least one typical antipsychotic agent in the manufacture of a medicament for adjunctive therapeutic administration for the treatment of cognitive impairment in schizophrenia in a patient receiving therapeutic administration of a functional muscarinic M1/M4 receptor agonist or a pharmaceutically acceptable salt thereof, in particular sabcomeline or a pharmaceutically acceptable salt thereof.

In a further aspect, the invention provides the use of at least one atypical antipsychotic agent in the manufacture of a medicament for adjunctive therapeutic administration for the treatment of cognitive impairment in schizophrenia in a patient receiving therapeutic administration of a functional muscarinic M1/M4 receptor agonist or a pharmaceutically acceptable salt thereof, in particular sabcomeline or a pharmaceutically acceptable salt thereof.

The invention also provides the use of at least one neuroprotective and/or neuroleptic agent (which may be a typical or atypical antipsychotic agent) for the treatment of cognitive impairment in schizophrenia in a patient receiving therapeutic administration of a functional muscarinic M1/M4 receptor agonist or a pharmaceutically acceptable salt thereof, in particular sabcomeline or a pharmaceutically acceptable salt thereof.

The invention also provides the use of at least one neuroprotective agent for the treatment of cognitive impairment in schizophrenia in a patient receiving therapeutic administration of a functional muscarinic M1/M4 receptor agonist or a pharmaceutically acceptable salt thereof, in particular sabcomeline or a pharmaceutically acceptable salt thereof.

The invention also provides the use of at least one neuroleptic agent for the treatment of cognitive impairment in schizophrenia in a patient receiving therapeutic administration of a functional muscarinic M1/M4 receptor agonist or a pharmaceutically acceptable salt thereof, in particular sabcomeline or a pharmaceutically acceptable salt thereof.

The invention also provides the use of at least one typical antipsychotic agent for the treatment of cognitive impairment in schizophrenia in a patient receiving therapeutic administration of a functional muscarinic M1/M4 receptor agonist or a pharmaceutically acceptable salt thereof, in particular sabcomeline or a pharmaceutically acceptable salt thereof.

The invention also provides the use of at least one atypical antipsychotic agent for the treatment of cognitive impairment in schizophrenia in a patient receiving therapeutic administration of a functional muscarinic M1/M4 receptor agonist or a pharmaceutically acceptable salt thereof, in particular sabcomeline or a pharmaceutically acceptable salt thereof.

In a further aspect, the invention provides a method of treatment of cognitive impairment in schizophrenia by simultaneous therapeutic administration of the functional muscarinic M1/M4 receptor agonist or a pharmaceutically acceptable salt thereof, in particular sabcomeline or a pharmaceutically acceptable salt thereof in combination with at least one neuroprotective agent and/or neuroleptic agent (which may be a typical or atypical antipsychotic agent).

In a further aspect, the invention provides a method of treatment of cognitive impairment in schizophrenia by simultaneous therapeutic administration of the functional muscarinic M1/M4 receptor agonist or a pharmaceutically acceptable salt thereof, in particular sabcomeline or a pharmaceutically acceptable salt thereof in combination with at least one neuroprotective agent.

In a further aspect, the invention provides a method of treatment of cognitive impairment in schizophrenia by simultaneous therapeutic administration of the functional muscarinic M1/M4 receptor agonist or a pharmaceutically acceptable salt thereof, in particular sabcomeline or a pharmaceutically acceptable salt thereof in combination with at least one neuroleptic agent.

In a further aspect, the invention provides a method of treatment of cognitive impairment in schizophrenia by simultaneous therapeutic administration of the functional muscarinic M1/M4 receptor agonist or a pharmaceutically acceptable salt thereof, in particular sabcomeline or a pharmaceutically acceptable salt thereof in combination with at least one typical antipsychotic agent.

In a further aspect, the invention provides a method of treatment of cognitive impairment in schizophrenia by simultaneous therapeutic administration of the functional muscarinic M1/M4 receptor agonist or a pharmaceutically acceptable salt thereof, in particular sabcomeline or a pharmaceutically acceptable salt thereof in combination with at least one atypical antipsychotic agent.

The invention further provides the use of a combination of a functional muscarinic M1/M4 receptor agonist or a pharmaceutically acceptable salt thereof, in particular sabcomeline or a pharmaceutically acceptable salt thereof and at least one neuroprotective agent and/or neuroleptic agent (which may be a typical or atypical antipsychotic agent) in the manufacture of a medicament for simultaneous therapeutic administration in the treatment of cognitive impairment in schizophrenia.

The invention further provides the use of a combination of a functional muscarinic M1/M4 receptor agonist or a pharmaceutically acceptable salt thereof, in particular sabcomeline or a pharmaceutically acceptable salt thereof and at least one neuroprotective agent in the manufacture of a medicament for simultaneous therapeutic administration in the treatment of cognitive impairment in schizophrenia.

The invention further provides the use of a combination of a functional muscarinic M1/M4 receptor agonist or a pharmaceutically acceptable salt thereof, in particular sabcomeline or a pharmaceutically acceptable salt thereof and at least one neuroleptic agent in the manufacture of a medicament for simultaneous therapeutic administration in the treatment of cognitive impairment in schizophrenia.

The invention further provides the use of a combination of a functional muscarinic M1/M4 receptor agonist or a pharmaceutically acceptable salt thereof, in particular sabcomeline or a pharmaceutically acceptable salt thereof and at least one typical antipsychotic agent in the manufacture of a medicament for simultaneous therapeutic administration in the treatment of cognitive impairment in schizophrenia.

The invention further provides the use of a combination of a functional muscarinic M1/M4 receptor agonist or a pharmaceutically acceptable salt thereof, in particular sabcomeline or a pharmaceutically acceptable salt thereof and at least one atypical antipsychotic agent in the manufacture of a medicament for simultaneous therapeutic administration in the treatment of cognitive impairment in schizophrenia.

The invention further provides the use of a combination of a functional muscarinic M1/M4 receptor agonist or a pharmaceutically acceptable salt thereof, in particular sabcomeline or a pharmaceutically acceptable salt thereof and at least one neuroprotective agent and/or neuroleptic agent (which may be a typical or atypical antipsychotic agent) for simultaneous therapeutic administration in the treatment of cognitive impairment in schizophrenia.

The invention further provides the use of a combination of a functional muscarinic M1/M4 receptor agonist or a pharmaceutically acceptable salt thereof, in particular sabcomeline or a pharmaceutically acceptable salt thereof and at least one neuroprotective agent for simultaneous therapeutic administration in the treatment of cognitive impairment in schizophrenia.

The invention further provides the use of a combination of a functional muscarinic M1/M4 receptor agonist or a pharmaceutically acceptable salt thereof, in particular sabcomeline or a pharmaceutically acceptable salt thereof and at least one neuroleptic agent for simultaneous therapeutic administration in the treatment of cognitive impairment in schizophrenia.

The invention further provides the use of a combination of a functional muscarinic M1/M4 receptor agonist or a pharmaceutically acceptable salt thereof, in particular sabcomeline or a pharmaceutically acceptable salt thereof and at least one typical antipsychotic agent for simultaneous therapeutic administration in the treatment of cognitive impairment in schizophrenia.

The invention further provides the use of a combination of a functional muscarinic M1/M4 receptor agonist or a pharmaceutically acceptable salt thereof, in particular sabcomeline or a pharmaceutically acceptable salt thereof and at least one atypical antipsychotic agent for simultaneous therapeutic administration in the treatment of cognitive impairment in schizophrenia.

The invention further provides the use of a functional muscarinic M1/M4 receptor agonist or a pharmaceutically acceptable salt thereof, in particular sabcomeline or a pharmaceutically acceptable salt thereof in the manufacture of a medicament for simultaneous therapeutic administration with at least neuroprotective agent and/or neuroleptic agent (which may be a typical or atypical antipsychotic agent) in the treatment of cognitive impairment in schizophrenia.

The invention further provides the use of a functional muscarinic M1/M4 receptor agonist or a pharmaceutically acceptable salt thereof, in particular sabcomeline or a pharmaceutically acceptable salt thereof in the manufacture of a medicament for simultaneous therapeutic administration with at least one neuroprotective agent in the treatment of cognitive impairment in schizophrenia.

The invention further provides the use of a functional muscarinic M1/M4 receptor agonist or a pharmaceutically acceptable salt thereof, in particular sabcomeline or a pharmaceutically acceptable salt thereof in the manufacture of a medicament for simultaneous therapeutic administration with at least one neuroleptic agent in the treatment of cognitive impairment in schizophrenia.

The invention further provides the use of a functional muscarinic M1/M4 receptor agonist or a pharmaceutically acceptable salt thereof, in particular sabcomeline or a pharmaceutically acceptable salt thereof in the manufacture of a medicament for simultaneous therapeutic administration with at least one atypical antipsychotic agent in the treatment of cognitive impairment in schizophrenia.

The invention further provides the use of a functional muscarinic M1/M4 receptor agonist or a pharmaceutically acceptable salt thereof, in particular sabcomeline or a pharmaceutically acceptable salt thereof for simultaneous therapeutic administration with at least one neuroprotective agent and/or neuroleptic agent (which may be a typical or atypical antipsychotic agent) in the treatment of cognitive impairment in schizophrenia.

The invention further provides the use of a functional muscarinic M1/M4 receptor agonist or a pharmaceutically acceptable salt thereof, in particular sabcomeline or a pharmaceutically acceptable salt thereof for simultaneous therapeutic administration with at least one neuroprotective agent in the treatment of cognitive impairment in schizophrenia.

The invention further provides the use of a functional muscarinic M1/M4 receptor agonist or a pharmaceutically acceptable salt thereof, in particular sabcomeline or a pharmaceutically acceptable salt thereof for simultaneous therapeutic administration with at least one neuroleptic agent in the treatment of cognitive impairment in schizophrenia.

The invention further provides the use of a functional muscarinic M1/M4 receptor agonist or a pharmaceutically acceptable salt thereof, in particular sabcomeline or a pharmaceutically acceptable salt thereof for simultaneous therapeutic administration with at least one typical antipsychotic agent in the treatment of cognitive impairment in schizophrenia.

The invention further provides the use of a functional muscarinic M1/M4 receptor agonist or a pharmaceutically acceptable salt thereof, in particular sabcomeline or a pharmaceutically acceptable salt thereof for simultaneous therapeutic administration with at least one atypical antipsychotic agent in the treatment of cognitive impairment in schizophrenia.

The invention further provides a functional muscarinic M1/M4 receptor agonist or a pharmaceutically acceptable salt thereof, in particular sabcomeline or a pharmaceutically acceptable salt thereof for use for simultaneous therapeutic administration with at least one neuroprotective agent and/or neuroleptic agent (which may be a typical or atypical antipsychotic agent) in the treatment of cognitive impairment in schizophrenia.

The invention further provides a functional muscarinic M1/M4 receptor agonist or a pharmaceutically acceptable salt thereof, in particular sabcomeline or a pharmaceutically acceptable salt thereof for use for simultaneous therapeutic administration with at least one neuroprotective agent in the treatment of cognitive impairment in schizophrenia.

The invention further provides a functional muscarinic M1/M4 receptor agonist or a pharmaceutically acceptable salt thereof, in particular sabcomeline or a pharmaceutically acceptable salt thereof for use for simultaneous therapeutic administration with at least one neuroleptic agent in the treatment of cognitive impairment in schizophrenia.

The invention further provides a functional muscarinic M1/M4 receptor agonist or a pharmaceutically acceptable salt thereof, in particular sabcomeline or a pharmaceutically acceptable salt thereof for use for simultaneous therapeutic administration with at least one typical antipsychotic agent in the treatment of cognitive impairment in schizophrenia.

The invention further provides a functional muscarinic M1/M4 receptor agonist or a pharmaceutically acceptable salt thereof, in particular sabcomeline or a pharmaceutically acceptable salt thereof for use for simultaneous therapeutic administration with at least one atypical antipsychotic agent in the treatment of cognitive impairment in schizophrenia.

The invention further provides the use of at least one neuroprotective agent and/or neuroleptic agent (which may be a typical or atypical antipsychotic agent) in the manufacture of a medicament for simultaneous therapeutic administration with a functional muscarinic M1/M4 receptor agonist or a pharmaceutically acceptable salt thereof, in particular sabcomeline or a pharmaceutically acceptable salt thereof in the treatment of cognitive impairment in schizophrenia.

The invention further provides the use of at least one neuroprotective agent in the manufacture of a medicament for simultaneous therapeutic administration with a functional muscarinic M1/M4 receptor agonist or a pharmaceutically acceptable salt thereof, in particular sabcomeline or a pharmaceutically acceptable salt thereof in the treatment of cognitive impairment in schizophrenia.

The invention further provides the use of at least one neuroleptic agent in the manufacture of a medicament for simultaneous therapeutic administration with a functional muscarinic M1/M4 receptor agonist or a pharmaceutically acceptable salt thereof, in particular sabcomeline or a pharmaceutically acceptable salt thereof in the treatment of cognitive impairment in schizophrenia.

The invention further provides the use of at least one typical antipsychotic agent in the manufacture of a medicament for simultaneous therapeutic administration with a functional muscarinic M1/M4 receptor agonist or a pharmaceutically acceptable salt thereof, in particular sabcomeline or a pharmaceutically acceptable salt thereof in the treatment of cognitive impairment in schizophrenia.

The invention further provides the use of at least one atypical antipsychotic agent in the manufacture of a medicament for simultaneous therapeutic administration with a functional muscarinic M1/M4 receptor agonist or a pharmaceutically acceptable salt thereof, in particular sabcomeline or a pharmaceutically acceptable salt thereof in the treatment of cognitive impairment in schizophrenia.

The invention further provides the use of at least one neuroprotective agent and/or neuroleptic agent (which may be a typical or atypical antipsychotic agent) for simultaneous therapeutic administration with a functional muscarinic M1/M4 receptor agonist or a pharmaceutically acceptable salt thereof, in particular sabcomeline or a pharmaceutically acceptable salt thereof in the treatment of cognitive impairment in schizophrenia.

The invention further provides the use of at least one neuroprotective agent for simultaneous therapeutic administration with a functional muscarinic M1/M4 receptor agonist or a pharmaceutically acceptable salt thereof, in particular sabcomeline or a pharmaceutically acceptable salt thereof in the treatment of cognitive impairment in schizophrenia.

The invention further provides the use of at least one neuroleptic agent for simultaneous therapeutic administration with a functional muscarinic M1/M4 receptor agonist or a pharmaceutically acceptable salt thereof, in particular sabcomeline or a pharmaceutically acceptable salt thereof in the treatment of cognitive impairment in schizophrenia.

The invention further provides the use of at least one typical antipsychotic agent for simultaneous therapeutic administration with a functional muscarinic M1/M4 receptor agonist or a pharmaceutically acceptable salt thereof, in particular sabcomeline or a pharmaceutically acceptable salt thereof in the treatment of cognitive impairment in schizophrenia.

The invention further provides the use of at least one atypical antipsychotic agent for simultaneous therapeutic administration with a functional muscarinic M1/M4 receptor agonist or a pharmaceutically acceptable salt thereof, in particular sabcomeline or a pharmaceutically acceptable salt thereof in the treatment of cognitive impairment in schizophrenia.

In further aspects, the invention provides

a method of treatment of cognitive impairment in schizophrenia by simultaneous therapeutic administration of a pharmaceutical composition comprising a functional muscarinic M1/M4 receptor agonist or a pharmaceutically acceptable salt thereof, in particular sabcomeline or a pharmaceutically acceptable salt thereof and at least one neuroprotective agent and/or neuroleptic agent (which may be a typical or atypical antipsychotic agent),
a pharmaceutical composition comprising a functional muscarinic M1/M4 receptor agonist or a pharmaceutically acceptable salt thereof, sabcomeline or a pharmaceutically acceptable salt thereof and at least one neuroprotective agent and/or neuroleptic agent (which may be a typical or atypical antipsychotic agent),

the use of a pharmaceutical composition comprising a functional muscarinic M1/M4 receptor agonist or a pharmaceutically acceptable salt thereof, in particular sabcomeline or a pharmaceutically acceptable salt thereof and at least one neuroprotective agent and/or neuroleptic agent (which may be a typical or atypical antipsychotic agent) for the treatment of cognitive impairment in schizophrenia,

the use of a pharmaceutical composition comprising a functional muscarinic M1/M4 receptor agonist or a pharmaceutically acceptable salt thereof, in particular sabcomeline or a pharmaceutically acceptable salt thereof and at least one neuroprotective agent and/or neuroleptic agent (which may be a typical or atypical antipsychotic agent), in the manufacture of a medicament for the treatment of cognitive impairment in schizophrenia, and
a pharmaceutical composition comprising a functional muscarinic M1/M4 receptor agonist or a pharmaceutically acceptable salt thereof, in particular sabcomeline or a pharmaceutically acceptable salt thereof and at least one neuroprotective agent and/or neuroleptic agent (which may be a typical or atypical antipsychotic agent) for use in the treatment of cognitive impairment in schizophrenia.

In further aspects, the invention provides

a method of treatment of cognitive impairment in schizophrenia by simultaneous therapeutic administration of a pharmaceutical composition comprising a functional muscarinic M1/M4 receptor agonist or a pharmaceutically acceptable salt thereof, in particular sabcomeline or a pharmaceutically acceptable salt thereof and at least one neuroprotective agent,
a pharmaceutical composition comprising a functional muscarinic M1/M4 receptor agonist or a pharmaceutically acceptable salt thereof, sabcomeline or a pharmaceutically acceptable salt thereof and at least one neuroprotective agent,
the use of a pharmaceutical composition comprising a functional muscarinic M1/M4 receptor agonist or a pharmaceutically acceptable salt thereof, in particular sabcomeline or a pharmaceutically acceptable salt thereof and at least one neuroprotective agent for the treatment of cognitive impairment in schizophrenia,
the use of a pharmaceutical composition comprising a functional muscarinic M1/M4 receptor agonist or a pharmaceutically acceptable salt thereof, in particular sabcomeline or a pharmaceutically acceptable salt thereof and at least one neuroprotective agent, in the manufacture of a medicament for the treatment of cognitive impairment in schizophrenia, and
a pharmaceutical composition comprising a functional muscarinic M1/M4 receptor agonist or a pharmaceutically acceptable salt thereof, in particular sabcomeline or a pharmaceutically acceptable salt thereof and at least one neuroprotective agent for use in the treatment of cognitive impairment in schizophrenia.

In further aspects, the invention provides

a method of treatment of cognitive impairment in schizophrenia by simultaneous therapeutic administration of a pharmaceutical composition comprising a functional muscarinic M1/M4 receptor agonist or a pharmaceutically acceptable salt thereof, in particular sabcomeline or a pharmaceutically acceptable salt thereof and at least one neuroleptic agent,
a pharmaceutical composition comprising a functional muscarinic M1/M4 receptor agonist or a pharmaceutically acceptable salt thereof, sabcomeline or a pharmaceutically acceptable salt thereof and at least one neuroleptic agent,
the use of a pharmaceutical composition comprising a functional muscarinic M1/M4 receptor agonist or a pharmaceutically acceptable salt thereof, in particular sabcomeline or a pharmaceutically acceptable salt thereof and at least one neuroleptic agent for the treatment of cognitive impairment in schizophrenia,
the use of a pharmaceutical composition comprising a functional muscarinic M1/M4 receptor agonist or a pharmaceutically acceptable salt thereof, in particular sabcomeline or a pharmaceutically acceptable salt thereof and at least one neuroleptic agent, in the manufacture of a medicament for the treatment of cognitive impairment in schizophrenia, and
a pharmaceutical composition comprising a functional muscarinic M1/M4 receptor agonist or a pharmaceutically acceptable salt thereof, in particular sabcomeline or a pharmaceutically acceptable salt thereof and at least one neuroleptic agent for use in the treatment of cognitive impairment in schizophrenia.

In further aspects, the invention provides

a method of treatment of cognitive impairment in schizophrenia by simultaneous therapeutic administration of a pharmaceutical composition comprising a functional muscarinic M1/M4 receptor agonist or a pharmaceutically acceptable salt thereof, in particular sabcomeline or a pharmaceutically acceptable salt thereof and at least one typical antipsychotic agent,
a pharmaceutical composition comprising a functional muscarinic M1/M4 receptor agonist or a pharmaceutically acceptable salt thereof, sabcomeline or a pharmaceutically acceptable salt thereof and at least one typical antipsychotic agent,
the use of a pharmaceutical composition comprising a functional muscarinic M1/M4 receptor agonist or a pharmaceutically acceptable salt thereof, in particular sabcomeline or a pharmaceutically acceptable salt thereof and at least one typical antipsychotic agent for the treatment of cognitive impairment in schizophrenia,
the use of a pharmaceutical composition comprising a functional muscarinic M1/M4 receptor agonist or a pharmaceutically acceptable salt thereof, in particular sabcomeline or a pharmaceutically acceptable salt thereof and at least one typical antipsychotic agent, in the manufacture of a medicament for the treatment of cognitive impairment in schizophrenia, and
a pharmaceutical composition comprising a functional muscarinic M1/M4 receptor agonist or a pharmaceutically acceptable salt thereof, in particular sabcomeline or a pharmaceutically acceptable salt thereof and at least one typical antipsychotic agent for use in the treatment of cognitive impairment in schizophrenia.

In further aspects, the invention provides

a method of treatment of cognitive impairment in schizophrenia by simultaneous therapeutic administration of a pharmaceutical composition comprising a functional muscarinic M1/M4 receptor agonist or a pharmaceutically acceptable salt thereof, in particular sabcomeline or a pharmaceutically acceptable salt thereof and at least one atypical antipsychotic agent,
a pharmaceutical composition comprising a functional muscarinic M1/M4 receptor agonist or a pharmaceutically acceptable salt thereof, sabcomeline or a pharmaceutically acceptable salt thereof and at least one atypical antipsychotic agent,
the use of a pharmaceutical composition comprising a functional muscarinic M1/M4 receptor agonist or a pharmaceutically acceptable salt thereof, in particular sabcomeline or a pharmaceutically acceptable salt thereof and at least one atypical antipsychotic agent for the treatment of cognitive impairment in schizophrenia,
the use of a pharmaceutical composition comprising a functional muscarinic M1/M4 receptor agonist or a pharmaceutically acceptable salt thereof, in particular sabcomeline or a pharmaceutically acceptable salt thereof and at least one atypical antipsychotic agent, in the manufacture of a medicament for the treatment of cognitive impairment in schizophrenia, and
a pharmaceutical composition comprising a functional muscarinic M1/M4 receptor agonist or a pharmaceutically acceptable salt thereof, in particular sabcomeline or a pharmaceutically acceptable salt thereof and at least one atypical antipsychotic agent for use in the treatment of cognitive impairment in schizophrenia.

In a further aspect, the invention provides a kit for use in the treatment of cognitive impairment in schizophrenia comprising a first dosage form comprising a functional muscarinic M1/M4 receptor agonist or a pharmaceutically acceptable salt thereof, in particular sabcomeline or a pharmaceutically acceptable salt thereof and one or more further dosage forms each comprising a neuroprotective agent and/or neuroleptic agent (which may be a typical or atypical antipsychotic agent) for simultaneous therapeutic administration.

In a further aspect, the invention provides a kit for use in the treatment of cognitive impairment in schizophrenia comprising a first dosage form comprising a functional muscarinic M1/M4 receptor agonist or a pharmaceutically acceptable salt thereof, in particular sabcomeline or a pharmaceutically acceptable salt thereof and one or more further dosage forms each comprising a neuroprotective agent for simultaneous therapeutic administration.

In a further aspect, the invention provides a kit for use in the treatment of cognitive impairment in schizophrenia comprising a first dosage form comprising a functional muscarinic M1/M4 receptor agonist or a pharmaceutically acceptable salt thereof, in particular sabcomeline or a pharmaceutically acceptable salt thereof and one or more further dosage forms each comprising a neuroleptic agent for simultaneous therapeutic administration.

In a further aspect, the invention provides a kit for use in the treatment of cognitive impairment in schizophrenia comprising a first dosage form comprising a functional muscarinic M1/M4 receptor agonist or a pharmaceutically acceptable salt thereof, in particular sabcomeline or a pharmaceutically acceptable salt thereof and one or more further dosage forms each comprising a typical antipsychotic agent for simultaneous therapeutic administration.

In a further aspect, the invention provides a kit for use in the treatment of cognitive impairment in schizophrenia comprising a first dosage form comprising a functional muscarinic M1/M4 receptor agonist or a pharmaceutically acceptable salt thereof, in particular sabcomeline or a pharmaceutically acceptable salt thereof and one or more further dosage forms each comprising an atypical antipsychotic agent for simultaneous therapeutic administration.

A neuroprotective agent may be defined as a compound which is intended to help limit the damage suffered by a nerve or neural tissue such as, for example, spinal cord, brain or nerve, when the blood supply is cut off or there is a traumatic injury. It is envisaged that psychotic disorders or diseases may be due in part to the abnormalities of neurons or synaptic function or architecture such as to cause a breakdown of neural integrity. It is believed that neuroprotective agents help prevent or stop the breakdown of neurons and neural integrity. Administering a neuroprotective agent alters the underlying pathology affecting integrity of neural function.

Neuroprotective agents include, but are not limited to, some types of antioxidants, antiinflammatories and anti-manic/mood stabiliser agents such as lithium. Preferably, the neuroprotective agent is selected from the group consisting of anti-oxidants, for example Vitamin E, eicosapentaenoic acid (EPA), docosahexaenoic acid (DHA), and anti-inflammatories such as non-steroidal anti-inflammatory, cyclo-oxygenase-2 (cox-2) inhibitors and statins.

When the chosen neuroprotective agent is also an antipsychotic (typical or atypical), it is believed that the clinical utility of the combination of sabcomeline and antipsychotic may vary between different members of the atypical antipsychotic drug class, depending on their different affinities for various sub-types of neurochemical receptors. For example, in addition to their affinities for dopamine and serotonin receptors, members of the atypical antipsychotic class may vary in their affinity for muscarinic and histamine receptor subtypes.

The activity of atypical antipsychotics at muscarinic receptor subtypes are such that properties of negligible affinity, weak agonist activity and weak antagonist activity have been reported amongst the various members of the atypical antipsychotic drug class.

As an example, the M1/M4 receptor agonist properties of sabcomeline may enhance functional cholinergic activity and, when administered in combination, provide benefit by:
i) enhancing functional cholinergic activity in combination with an atypical antipsychotic that itself has little or no affinity for muscarinic receptors (e.g. risperidone);
ii) providing additive functional cholinergic activity in combination with an atypical antipsychotic drug that has weak muscarinic receptor agonist effects (e.g. clozapine or N-desmethylclozapine);
iii) competing for muscarinic receptors and thereby reducing the anticholinergic functional effects of an atypical antipsychotic drug that possesses muscarinic receptor antagonist properties (e.g. olanzapine).

As well as muscarinic and histaminergic receptors there are other mechanisms that may have beneficial or adverse effects on cognition. For instance drugs with 5-HT6 receptor antagonist and an adrenergic α2 receptor antagonist properties may also be of benefit. Some atypical antipsychotics also have these benefits.

A particular example of a neuroprotective agent useful in the invention, its typical route of administration and dosage ranges that are preferred is lithium, trade name Priadel, oral tablet or liquid, 100-1000 gms titrated to plasma level

The term neuroleptic refers to drugs which have the effect on cognition and behaviour of antipsychotic drugs that reduce confusion, delusions, hallucinations, and psychomotor agitation in patients with psychoses. Also known as major tranquilizers and antipsychotic drugs, neuroleptic agents include, but are not limited to:

typical antipsychotic drugs, including phenothiazines, further divided into the aliphatics, piperidines, and piperazines, thioxanthenes (e.g., droperidol), butyrophenones (e.g. haloperidol), dibenzoxazepines (e.g. loxapine), dihydroindolones (e.g. molindone), and diphenylbutylpiperidines (e.g. pimozide), and.
atypical antipsychotic drugs, including benzisoxazoles (e.g. risperidone), olanzapine, quetiapine, osanetant and ziprasidone.

A particular example of a neuroleptic agent useful in the invention and its typical route of administration and dosage ranges that are preferred is olanzapine, trade name Zyprexa, oral tablet, 5 to 20 mg

Particularly preferred neuroleptic agents for use in the invention are olanzapine, risperidone, quetiapine, aripiprazole, haloperidol, clozapine, ziprasidone and osanetant.

The functional muscarinic M1/M4 receptor agonist or a pharmaceutically acceptable salt thereof, in particular sabcomeline or a pharmaceutically acceptable salt thereof, neuroprotective agent and/or neuroleptic agent (which may be a typical or atypical antipsychotic), may be administered, by weight, in the ranges 10 μg-200 μg, 0.0-5 μg, 0.0-5 μg and 0.0-5 μg respectively. Such dosages are typically given once every 12 hours for 3 weeks before being re-assessed by a physician.

Preferably, the functional muscarinic M1/M4 receptor agonist or a pharmaceutically acceptable salt thereof, in particular sabcomeline or a pharmaceutically acceptable salt thereof is administered in the range between 20 μg-100 μg. More preferably, the functional muscarinic M1/M4 receptor agonist or a pharmaceutically acceptable salt thereof, in particular sabcomeline or a pharmaceutically acceptable salt thereof is administered in the range between 25 μg-50 μg.

The combination therapies of the invention are preferably administered adjunctively. By adjunctive administration is meant the concurrent or overlapping administration of each of the components in the form of separate pharmaceutical compositions or devices. This regime of therapeutic administration of two or more therapeutic agents is referred to generally by those skilled in the art and herein as adjunctive therapeutic administration; it is also known as add-on therapeutic administration.

Any and all treatment regimes in which a patient receives separate but concurrent or overlapping therapeutic administration of the functional muscarinic M1/M4 receptor agonist or a pharmaceutically acceptable salt thereof, in particular sabcomeline or a pharmaceutically acceptable salt thereof and at least one neuroprotective and/or neuroleptic agent (which may be a typical or atypical antipsychotic agent) are within the scope of the current invention. In one embodiment of adjunctive therapeutic administration as described herein, a patient is typically stabilised on a therapeutic administration of one or more of the components for a period of time and then receives administration of another component.

Within the scope of this invention, it is preferred that the functional muscarinic M1/M4 receptor agonist or a pharmaceutically acceptable salt thereof, in particular sabcomeline or a pharmaceutically acceptable salt thereof is administered as adjunctive therapeutic treatment to patients who are receiving administration of at least one
a) neuroprotective agent, or
b) neuroleptic agent (which may be a typical or atypical antipsychotic agent).

However, the scope of the invention also includes the adjunctive therapeutic administration of at least one neuroprotective and/or neuroleptic agent (which may be a typical or atypical antipsychotic agent) to patients who are receiving administration of the functional muscarinic M1/M4 receptor agonist or a pharmaceutically acceptable salt thereof, in particular sabcomeline or a pharmaceutically acceptable salt thereof.

The combination therapies of the invention may also be administered simultaneously. By simultaneous administration is meant a treatment regime wherein the individual components are administered together, either in the form of a single pharmaceutical composition or device comprising or containing two or more components, or as separate compositions or devices, each comprising one of the components, administered simultaneously. Such combinations of the separate individual components for simultaneous combination may be provided in the form of a kit.

The treatment of cognitive impairment in schizophrenia with the functional muscarinic M1/M4 receptor agonist or a pharmaceutically acceptable salt thereof, in particular sabcomeline or a pharmaceutically acceptable salt thereof or with the functional muscarinic M1/M4 receptor agonist or a pharmaceutically acceptable salt thereof, in particular sabcomeline or a pharmaceutically acceptable salt thereof and at least one neuroprotective agent and/or neuroleptic agent (which may be a typical or atypical antipsychotic agent) as defined in the present invention may occur in addition to further drug therapies.

The functional muscarinic M1/M4 receptor agonist or a pharmaceutically acceptable salt thereof, in particular sabcomeline or a pharmaceutically acceptable salt thereof may also be used in various combined ways with at least one neuroprotective agent and/or neuroleptic agent (which may be a typical or atypical antipsychotic).

In a further aspect, the invention provides a pharmaceutical composition comprising
a) a functional muscarinic M1/M4 receptor agonist or a pharmaceutically acceptable salt thereof, in particular sabcomeline or a pharmaceutically acceptable salt thereof and
b) one or more pharmaceutically acceptable excipients.

The composition may also comprise one neuroprotective and/or neuroleptic agent (which may be a typical or atypical antipsychotic agent).

The invention thus provides the use of a pharmaceutical composition comprising the functional muscarinic M1/M4 receptor agonist or a pharmaceutically acceptable salt thereof, in particular sabcomeline or a pharmaceutically acceptable salt thereof and at least one neuroprotective and/or neuroleptic agent (which may be a typical or atypical antipsychotic agent) and one or more pharmaceutically acceptable excipients.

The invention thus provides the use of a pharmaceutical composition comprising the functional muscarinic M1/M4 receptor agonist or a pharmaceutically acceptable salt thereof, in particular sabcomeline or a pharmaceutically acceptable salt thereof and at least one neuroprotective and one or more pharmaceutically acceptable excipients.

The invention thus provides the use of a pharmaceutical composition comprising the functional muscarinic M1/M4 receptor agonist or a pharmaceutically acceptable salt thereof, in particular sabcomeline or a pharmaceutically acceptable salt thereof and at least one neuroleptic agent and one or more pharmaceutically acceptable excipients.

The invention thus provides the use of a pharmaceutical composition comprising the functional muscarinic M1/M4 receptor agonist or a pharmaceutically acceptable salt thereof, in particular sabcomeline or a pharmaceutically acceptable salt thereof and at least one typical antipsychotic agent and one or more pharmaceutically acceptable excipients.

The invention thus provides the use of a pharmaceutical composition comprising the functional muscarinic M1/M4 receptor agonist or a pharmaceutically acceptable salt thereof, in particular sabcomeline or a pharmaceutically acceptable salt thereof and at least one atypical antipsychotic agent and one or more pharmaceutically acceptable excipients.

For example, the pharmaceutical composition may comprise the functional muscarinic M1/M4 receptor agonist or a pharmaceutically acceptable salt thereof, in particular sabcomeline or a pharmaceutically acceptable salt thereof, neuroprotective agent and/or neuroleptic agent (which may be a typical or atypical antipsychotic agent), by weight, in the ranges 10 μg-200 μg, 0.0-5 μg, 0.0-5 μg and 0.0-5 μg respectively.

For example, the pharmaceutical composition may comprise the functional muscarinic M1/M4 receptor agonist or a pharmaceutically acceptable salt thereof, in particular sabcomeline or a pharmaceutically acceptable salt thereof and neuroprotective agent, by weight, in the ranges 10 μg-200 μg and 0.0-5 μg respectively.

For example, the pharmaceutical composition may comprise the functional muscarinic M1/M4 receptor agonist or a pharmaceutically acceptable salt thereof, in particular sabcomeline or a pharmaceutically acceptable salt thereof and neuroleptic, by weight, in the ranges 10 μg-200 μg and 0.0-5 μg respectively.

For example, the pharmaceutical composition may comprise the functional muscarinic M1/M4 receptor agonist or a pharmaceutically acceptable salt thereof, in particular sabcomeline or a pharmaceutically acceptable salt thereof and typical antipsychotic agent, by weight, in the ranges 10 μg-200 μg and 0.0-5 μg respectively.

For example, the pharmaceutical composition may comprise the functional muscarinic M1/M4 receptor agonist or a pharmaceutically acceptable salt thereof, in particular sabcomeline or a pharmaceutically acceptable salt thereof and atypical antipsychotic agent, by weight, in the ranges 10 μg-200 μg and 0.0-5 μg respectively.

Such dosages are typically given once every 12 hours for 3 weeks before being re-assessed by a physician. Preferably, the functional muscarinic M1/M4 receptor agonist or a pharmaceutically acceptable salt thereof, in particular sabcomeline or a pharmaceutically acceptable salt thereof is present in the range between 20 μg-100 μg. More preferably, the functional muscarinic M1/M4 receptor agonist or a pharmaceutically acceptable salt thereof, in particular sabcomeline or a pharmaceutically acceptable salt thereof is present in the range between 25 μg-50 μg.

The choice of the most appropriate pharmaceutical compositions is within the skill of the art.

Suitable formulations include, but are not limited to tablets, capsules, powders, granules, lozenges, suppositories, reconstitutable powders, or liquid preparations such as oral or sterile parenteral solutions or suspensions.

It is preferred that the compositions used in the invention, are in the form of a unit dose.

Solid unit dose presentation forms for oral administration may be tablets and capsules and may contain conventional excipients such as binding agents, for example syrup, acacia, gelatin, sorbitol, tragacanth, or polyvinylpyrrolidone; fillers, for example lactose, sugar, maize-starch, calcium phosphate, sorbitol or glycine; tabletting lubricants, for example magnesium stearate; disintegrants, for example starch, polyvinylpyrrolidone, sodium starch glycollate or microcrystalline cellulose; or pharmaceutically acceptable wetting agents such as sodium lauryl sulphate.

The solid oral compositions may be prepared by conventional methods of blending, filling, tabletting or the like. Repeated blending operations may be used to distribute the active agent throughout those compositions employing large quantities of fillers. Such operations are of course conventional in the art. The tablets may be coated according to methods well known in normal pharmaceutical practice, in particular with an enteric coating.

Oral liquid preparations for use in the invention may be in the form of, for example, emulsions, syrups, suspensions or elixirs, or may be presented as a dry product for reconstitution with water or other suitable vehicle before use.

Such liquid preparations may contain conventional additives such as suspending agents, for example sorbitol, syrup, methyl cellulose, gelatin, hydroxyethylcellulose, carboxymethylcellulose, aluminium stearate gel, or hydrogenated edible fats; emulsifying agents, for example lecithin, sorbitan monooleate, or acacia; non-aqueous vehicles (which may include edible oils), for example almond oil, fractionated coconut oil, oily esters such as esters of glycerine, propylene glycol, or ethyl alcohol; preservatives, for example methyl or propyl phydroxybenzoate or sorbic acid; and if desired conventional flavouring or colouring agents.

For parenteral administration (for example intravenous, intravascular or subcutaneous administration) of compositions for use in the invention, fluid unit dosage forms are prepared utilizing the component or the combination of the components and a sterile vehicle, and, depending on the concentration used, can be either suspended or dissolved in the vehicle.

In preparing solutions the monotherapy component, the components of the combination therapy or the combination of the components can be dissolved in water for injection and filter sterilized before filling into a suitable vial or ampoule and sealing. Advantageously, adjuvants such as a local anaesthetic, a preservative and buffering agents can be dissolved in the vehicle. To enhance the stability, the composition can be frozen after filling into the vial and the water removed under vacuum.

Parenteral suspensions are prepared in substantially the same manner, except that the component is suspended in the vehicle instead of being dissolved, and sterilization cannot be accomplished by filtration.

The monotherapy component, the components of the combination therapy, or the combination of the components can be sterilized by exposure to ethylene oxide before suspending in the sterile vehicle. Advantageously, a surfactant or wetting agent is included in the composition to facilitate uniform distribution of the component or the combination of the components.

Alternatively, the components may be prepared in solid form which melts on contact with the tongue of the patient, for example in the form of orally disintegrating tablets sold under the trade name ZYDIS®.

The compositions for use in the invention may be prepared as depot preparations. Such long acting formulations may be administered by implantation (for example subcutaneously or intramuscularly) or by intramuscular injection. Thus, for example, the monotherapy component, the components of the combination therapy or the combination of the components of the invention may be formulated with suitable polymeric or hydrophobic materials (for example as an emulsion in an acceptable oil) or ion exchange resins, or as sparingly soluble derivatives, for example, as a sparingly soluble salt.

The compositions for use in the invention may contain from 0.1% to 99% by weight, preferably from 10-60% by weight, of the active material, depending on the method of administration.

The unit dose of the functional muscarinic M1/M4 receptor agonist or a pharmaceutically acceptable salt thereof, in particular sabcomeline or a pharmaceutically acceptable salt thereof component in compositions according to the invention is in the range of 10 μg-200 μg. Preferably, the dose is between 20 μg-100 μg. More preferably, the dose is between 25 μg-50 μg. The dose may be administered as a single dose or twice daily. Ideally, sabcomeline is administered at a dose of 25 μg twice daily.

For the combination therapies, the daily and unit doses of the neuroprotective agent and/or neuroleptic agent (which may be a typical or atypical antipsychotic agent) will depend upon which neuroprotective agent and/or neuroleptic agent is employed, but may typically be the recommended or approved dosage for the specific neuroprotective agent and/or neuroleptic agent (which may be a typical or atypical antipsychotic agent when administered as monotherapy.

In a preferred aspect of the invention, adjunctive administration of the functional muscarinic M1/M4 receptor agonist or a pharmaceutically acceptable salt thereof, in particular sabcomeline or a pharmaceutically acceptable salt thereof may permit lower doses of the neuroprotective agent and/or neuroleptic agent (which may be a typical or atypical antipsychotic agent) than those normally recommended when the neuroprotective and/or neuroleptic agent is prescribed as monotherapy.

### EXAMPLE 1

An example of a method of preparation of sabcomeline is as follows: to a stirred solution of potassium tert-butoxide (94.1 g; 0.84 mol) in tetrahydrofuran (250 ml) under nitrogen is added a solution of 3-(cyanomethyl)quinucidine (60 g; 0.4 mol) in tetrahydrofuran (150 ml) during a period of 10 mins. The reaction is stirred for 10 minutes then cooled to 0øC. Isoamyl nitrite (51.5 g 0.44 mol) is added at a rate such that the internal temperature does not exceed 25øC. The reaction is stirred for 20 minutes then diluted with dimethylsulphoxide (500 ml).

Methyl tosylate (134 g; 0.72 mol) is added as a solution in dimethylsulphoxide (100 ml) at a rate such that the temperature does not exceed 35øC. After a further 20 minutes aqueous potassium carbonate (ca 5 wt % 500 ml) is added and the reaction extracted with ethyl acetate (5×200 ml). The ethyl acetate extract is washed with 5 wt % aqueous potassium carbonate (4×250 ml), then saturated potassium carbonate (50 ml). The combined aqueous layers are re-extracted with ethyl acetate (500 ml) which is washed as above. The combined organic extracts are dried over anhydrous potassium carbonate (200 g) and concentrated in vacuo to give a brown oil containing ca. 80 wt % 3-[(cyano)(methoxyimino)-methyl]quinuclidine as a 4:1 mixture of Z:E isomers, (47.4 g; 0.245 mol; 61%).

### EXAMPLE 2

The following patient study was a small Phase IIa, proof of concept, 51-day, multicentre, double-blind, placebo-controlled, rising dose parallel study of the efficacy and tolerability of sabcomeline in patients with acute exacerbation of chronic schizophrenia. A total of twenty eight patients, nineteen received sabcomeline and nine patients received placebo. Daily doses of sabcomeline were titrated from 50 μg daily through 100 μg to 150 μg daily over nine days.

The Primary Objective of the study was:
To assess the efficacy of sabcomeline in patients with schizophrenia (includes: effects on positive and negative symptoms of schizophrenia and general psychopathology)

The Secondary Objectives of the study were:
To assess the effects of sabcomeline on neurocognitive function in patients with schizophrenia
To study the safety and tolerability of sabcomeline in the treatment of patients with schizophrenia.

The results of the study showed the following:
1. No effect on PANSS positive symptoms: delusions, hallucinations
2. Benefit in PANSS general psychopathology: poor attention,
3. A trend in verbal memory (one area of the Rivermead Story test), per protocolanalysis.
4. A trend in selective attention and flexibility of thinking (one area of the Stroop Colour Test), per protocol analysis
5. A trend in attention and speed of processing (one area of Trail Making test B), per protocol analysis

This study was not powered to show statistical significance but rather to generate hypotheses about the potential efficacy of sabcomeline in the treatment of the positive, negative and cognitive symptoms of schizophrenia. Analysis of the data for 8 severe patients and 4 placebo set out below demonstrated that sabcomeline had benefit in the treatment of affective symptoms; cognitive impairment and change in behaviour.

TABLE
Positive and Negative syndrome Scale (PANSS) Item analysis on each
patient with at least moderate severity (≧4) at baseline

| | PANSS | | | |
|---|---|---|---|---|
| | Sabcomeline | | Placebo | |
| | Total N = 8 | | Total N = 4 | |
| | Baseline | Endpoint | Baseline | Endpoint |
| Positive Items | | | | |
| Delusions | 8 | 2/8 | 4 | 3/4 |
| Hallucinations | 4 | 2/4 | 4 | 4/4 |
| General | | | | |
| Psychopathology | | | | |
| Poor attention | 5 | 5/5 | 1 | 0/1 |
| Lack of judgment/insight | 4 | 1/4 | 4 | 1/4 |
| Poor volition | 4 | 4/4 | 1 | 0/1 |
| Preoccupation | 5 | 3/5 | 1 | 1/1 |
| Active social avoidance | 4 | 1/4 | 0 | 0 |

The safety and tolerability data for sabcomeline in this study are consistent with observations from clinical studies in other indications (see Example 3) which reveal a generally well-tolerated and safe compound.

### EXAMPLE 3

Sabcomeline 50 μg daily, or 25 μg twice daily, has also be evaluated in two 24-week placebo-controlled trials that included 880 patients with Alzheimer's disease. Sabcomeline was safe and well-tolerated across the dose range examined.

It will be appreciated, that sabcomeline is an example of a functional muscarinic M1/M4 receptor agonist which is used in the present invention. Other suitable functional M1/M4 receptor agonists or combinations thereof may also be used.

1.-50. (canceled)

51. A method of treatment of cognitive impairment in schizophrenia by administration of a functional muscarinic M1/M4 receptor agonist or a pharmaceutically acceptable salt thereof.

52. The method as claimed in claim 51, wherein the cognitive impairment is deficits in working memory, attention/vigilance, verbal learning and memory, visual learning and memory, speed of processing, reasoning and problem solving, and social cognition.

53. The method as claimed in claim 51, wherein the functional muscarinic M1/M4 receptor agonist is other than sabcomeline.

54. The method as claimed in claim 51, wherein said treatment comprises administering the functional muscarinic M1/M4 receptor agonist or a pharmaceutically acceptable salt thereof at a dose of between 10 μg-200 μg.

55. The method as claimed in claim 51, wherein said treatment comprises administering the functional muscarinic M1/M4 receptor agonist or a pharmaceutically acceptable salt thereof at a dose of between 25 μg-50 μg.

56. The method as claimed in claim 51, wherein said treatment comprises administering the functional muscarinic M1/M4 receptor agonist or a pharmaceutically acceptable salt thereof as a single dose or twice daily.

57. A method of treatment of cognitive impairment in schizophrenia by adjunctive therapeutic administration of a functional muscarinic M1/M4 receptor agonist or a pharmaceutically acceptable salt thereof to a patient receiving therapeutic administration of at least one neuroprotective agent and/or neuroleptic agent.

58. A method of treatment of cognitive impairment in schizophrenia by adjunctive therapeutic administration of a functional muscarinic M1/M4 receptor agonist or a pharmaceutically acceptable salt thereof to a patient receiving therapeutic administration of at least one typical antipsychotic agent,

59. A method of treatment of cognitive impairment in schizophrenia by adjunctive therapeutic administration of a functional muscarinic M1/M4 receptor agonist or a pharmaceutically acceptable salt thereof to a patient receiving therapeutic administration of at least one atypical antipsychotic agent.

60. The method as claimed in claim 57, wherein the neuroprotective agent is selected from the group consisting of anti-oxidants, eicosapentaenoic acid (EPA), docosahexaenoic acid (DHA), anti-inflammatories, cyclo-oxygenase-2 (cox-2) inhibitors, statins and lithium.

61. The method as claimed in claim 57, wherein the neuroleptic agent is selected from the group consisting of the following seven classes of drugs: phenothiazines, thioxanthenes, butyrophenones, dibenzoxazepines, dihydroindolones, diphenyl-butylpiperidines, and bezisoxazoles.

62. The method as claimed in claim 57, wherein the said treatment comprises administering the functional muscarinic M1/M4 receptor agonist or a pharmaceutically acceptable salt thereof, neuroprotective agent and/or neuroleptic agent, by percentage weight, in the ranges 1-100% and 0.0-99% respectively.

63. The method as claimed in claim 57, wherein the said treatment comprises administering the functional muscarinic M1/M4 receptor agonist or a pharmaceutically acceptable salt thereof, and typical antipsychotic agent, by percentage weight, in the ranges 1-100% and 0.0-99% respectively.

64. The method as claimed in claim 57, wherein the said treatment comprises administering the functional muscarinic M1/M4 receptor agonist or a pharmaceutically acceptable salt thereof, and atypical antipsychotic agent, by percentage weight, in the ranges 1-100% and 0.0-99% respectively.

65. The method as claimed in claim 57, wherein said treatment comprises administering the functional muscarinic M1/M4 receptor agonist or a pharmaceutically acceptable salt thereof, neuroprotective agent and/or neuroleptic agent, by weight, in the ranges 10 μg-200 μg, 0.0-5 μg, 0.0-5 μg and 0.0-5 μg respectively.

66. The method as claimed in claim 57, wherein said treatment comprises administering the functional muscarinic M1/M4 receptor agonist or a pharmaceutically acceptable salt thereof, and typical antipsychotic agent, by weight, in the ranges 10 μg-200 μg, 0.0-5 μg, 0.0-5 μg and 0.0-5 μg respectively.

67. The method as claimed in claim 57, wherein said treatment comprises administering the functional muscarinic M1/M4 receptor agonist or a pharmaceutically acceptable salt thereof, and atypical antipsychotic agent, by weight, in the ranges 10 μg-200 μg, 0.0-5 μg, 0.0-5 μg and 0.0-5 μg respectively.

68. A kit for use in the treatment of cognitive impairment in schizophrenia comprising a first dosage form comprising a functional muscarinic M1/M4 receptor agonist or a pharmaceutically acceptable salt thereof and at least one further dosage form comprising a neuroprotective agent and/or neuroleptic agent, for simultaneous therapeutic administration.

69. A kit for use in the treatment of cognitive impairment in schizophrenia comprising a first dosage form comprising a functional muscarinic M1/M4 receptor agonist or a pharmaceutically acceptable salt thereof and at least one further dosage form comprising a typical antipsychotic agent, for simultaneous therapeutic administration.

70. A kit for use in the treatment of cognitive impairment in schizophrenia comprising a first dosage form comprising a functional muscarinic M1/M4 receptor agonist or a pharmaceutically acceptable salt thereof and at least one further dosage form comprising an atypical antipsychotic agent, for simultaneous therapeutic administration.

71. The kit as claimed in claim 68, wherein the neuroprotective agent is selected from the group consisting of anti-oxidants, eicosapentaenoic acid (EPA), docosahexaenoic acid (DHA), anti-inflammatories, cyclo-oxygenase-2 (cox-2) inhibitors, statins and lithium

72. The kit as claimed in claim 68, wherein the neuroleptic agent is selected from the group consisting of the following seven classes of drugs:
phenothiazines, thioxanthenes, butyrophenones, dibenzoxazepines, dihydroindolones, diphenyl-butylpiperidines, and bezisoxazoles.

73. The kit as claimed in claim 68, wherein said kit comprises the functional muscarinic M1/M4 receptor agonist or a pharmaceutically acceptable salt thereof in a unit dose of between 10 μg-200 μg.
